(19) United States
(12) Patent Application Publication
(10) Pub. No.: US 2019/0046106 A1
(43) Pub. Date: Feb. 14, 2019

(54) PHOTOACOUSTIC DETECTION OF PSMA

(71) Applicants: Carlton R. Pennypacker, El Cerrito, CA (US); Anthony Carlis, Garrison Twp, MI (US)

(72) Inventors: Carlton R. Pennypacker, El Cerrito, CA (US); Anthony Carlis, Garrison Twp, MI (US)

(21) Appl. No.: 16/058,911

(22) Filed: Aug. 8, 2018

Related U.S. Application Data
(60) Provisional application No. 62/542,342, filed on Aug. 8, 2017.

(51) Int. Cl. A61B 5/00; A61B 5/06; A61B 17/32; A61K 49/22

(52) U.S. Cl. CPC A61B 5/4381; A61B 5/0095; A61B 5/745; A61B 5/6873; A61B 5/065; A61B 2017/00274; A61B 5/4836; A61K 49/221; A61B 2576/02; A61B 2017/320069; A61B 17/320016

(57) ABSTRACT

An apparatus for use in a minimally invasive prostate cancer detection system, using a fluorophore peptide dye conjugate compound which has at least one absorption wavelength in a range of 380 to 1400 nm, wherein said compound attaches to a prostate-specific membrane antigen (PSMA) expressed by a prostate cancer cell. A photo-acoustic imaging probe to be inserted in at least one of a rectum, urethra, or placed proximal the prostate. The probe having an emitter to emit a first signal at the prostrate and a prostate cancer, excite the conjugate compound and a receiver to receive a second signal from said conjugate compound, thereby indicating a cancerous region of the prostrate. A processor unit connected to said probe, is configured and operable for receiving and processing said to produce a tomographic representation of the prostrate.

### CROSS-REFERENCE TO RELATED APPLICATIONS

The subject patent application claims priority to and all the benefits of U.S. Provisional Patent Application No. 62/542,342, which was filed on Aug. 8, 2017, which is herein incorporated by reference in its entirety.

### FIELD OF INVENTION

The present invention relates generally to the fields of molecular biology and medicine. More particularly, it concerns imaging and the diagnosis and treatment of cancer with a focus on prostate cancer.

### BACKGROUND

In the United States, prostate cancer (PCa) has been the most commonly diagnosed cancer in males and is consistently among the leading causes of cancer-related deaths of men. According to the “2006 Cancer Facts and Figures” published by the American Cancer Society, an estimated 234,460 new cases of prostate cancer will be diagnosed and 27,350 men will die of prostate cancer in the United States alone in 2006. Most of the deaths from prostate cancer are related to an adjunct disease, in which patients present with bone metastasis and soft-tissue involvement.

The risk of Extraprostatic extension (EPE) and seminal vesicle invasion (SVI) are adverse prognostic factors in prostate cancer in patients with clinically localized disease typically remains at about 10% to 20%, despite definite local therapy. The skeleton is the most common site for metastases in a variety of cancers, among which breast and prostate cancers account for over 80% of cases causing the great morbidity due to intractable bone pain, pathological fractures, hypercalcemia and nerve compression. Once the tumor spreads to bone, it can become unresponsive to standard therapeutic treatments, and there is presently no effective treatment of bone metastases.

Therefore, the present invention describes concepts that hold the potential to provide an almost non-invasive early detection via a ultra-sound rectally inserted detection device which will show prostate cancer (and many other types of cancer) in many stages of its development. The present invention will detect tumors, for example, in a simplified model of tumor cell in which the tumor is a sphere at least as small as 1 mm diameter. Such a system and method can yield resolution for tumor detection with a 5× to 10× in linear dimension and 100× to 1000× smaller tumor volumes than detectable by contrast and implemented at a small fraction of the cost or imaging time of an MRI, or traditional TRUS. With current technology, resolutions of a 5 mm spherical cancer cell approximation are only possible. The present invention deploys techniques to increase the signal to noise ratio, such as signal averaging and a larger aperture, phased array system with image reconstruction.

Additionally, with breakthroughs in fluorophores carrying peptides that bond to nerve cells, thus permitting nerve protection during normal surgery or new methods enabled by the systems and methods described herein. If a surgeon can visualize nerves during prostate cancer elimination (either visual nerve imaging or photo-acoustic nerve imaging overlaid on prostate bed), the system holds the potential to significantly reduce nerve damage and facilitate very high quality care, scalable to many patients at minimal cost. Therefore, it would be beneficial to detect and remove any involved prostrate carcinomas before they can metastasize.

### BRIEF DESCRIPTION OF THE DRAWINGS

FIG. 1 is a schematic diagram of a photoacoustic probe detection system.

FIG. 2 is a schematic diagram of a second type of photoacoustic probe detection system.

FIG. 3 illustrates a photoacoustic probe.

FIG. 4 illustrates photoacoustic system implemented on a transportable cart.

FIG. 5 illustrates a front side (that presses against prostate) showing detail of photoacoustic probe head with an arrangement of integrated fiber optics transmitters and an arrangement of ultrasound receivers.

### DETAILED DESCRIPTION

Photo Acoustic Imaging

The photoacoustic effect was first discovered by Alexander Graham Bell. When a molecule absorbs a light particle, if enough molecules absorb enough light, an ultrasound pulse is emitted. That is, after absorption of optical light, the molecules in the medium heat up briefly, expand, and emit sound waves in the megahertz (Mhz) region of the electrical frequency spectrum.

For example, a laser probe may be inserted into the urethra and laser light from the laser probe from inside the urethra can excite a prostate cancer sticky peptide inserted into the subject, such a fluorophores which will then emit sound waves when the laser light is removed, thus permitting the identification and the location of the cancerous region of the prostrate. A peptide is any member of a class of compounds of low molecular weight that yield two or more amino acids on hydrolysis can be compounded to attach to a cancer cell, such as a prostate cancer cell or another cancer cell.

Alternatively, the normal rectal position of prostate ultrasound or trans rectal ultrasound (TRUS) probes when the urethra is too complex and small, and ultrasound probes through rectal access will be easier.

In addition, the tumor can be ablated for example, with a High intensity Focused Ultrasound (HIFU) tumor destruction via the probe. The probe, using beamforming via an array, can yield a focused beam at the tumor. The array permits the beam have varying apertures, so that the beam has very small side lobes, and does not do significant damage to healthy prostate tissue or nerves.

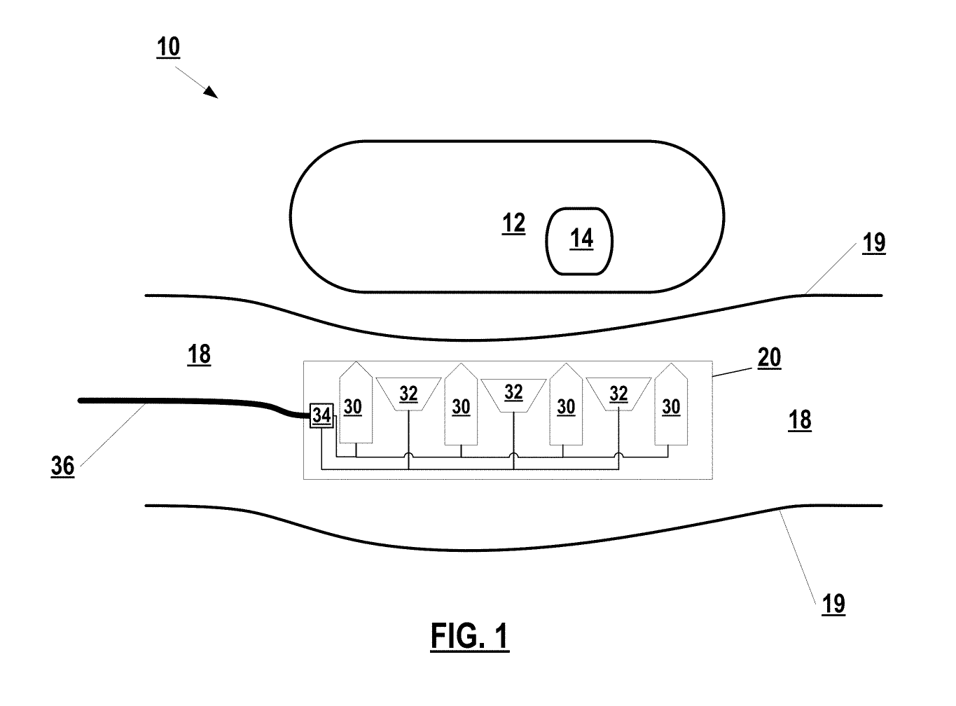

With reference to the Figures, wherein like numerals indicate like parts throughout the several views, FIG. 1 is schematic diagram of a photoacoustic probe detection system 10 for a minimally invasive prostate cancer detection system. A prostate 12 is shown with a cancerous region 14. A first probe 20 is inserted into a colon 19 going through a rectum 18 and placed adjacent to the prostate 12. The first probe 20 has an electronically steerable emitter 30 and an electronically steerable receiver 32. The electronically steerable emitter 30 can emit an energy pulse, such as a laser or other a radio frequency signal. The electronically steerable receiver 32 can be an array of ultra sound sensors.

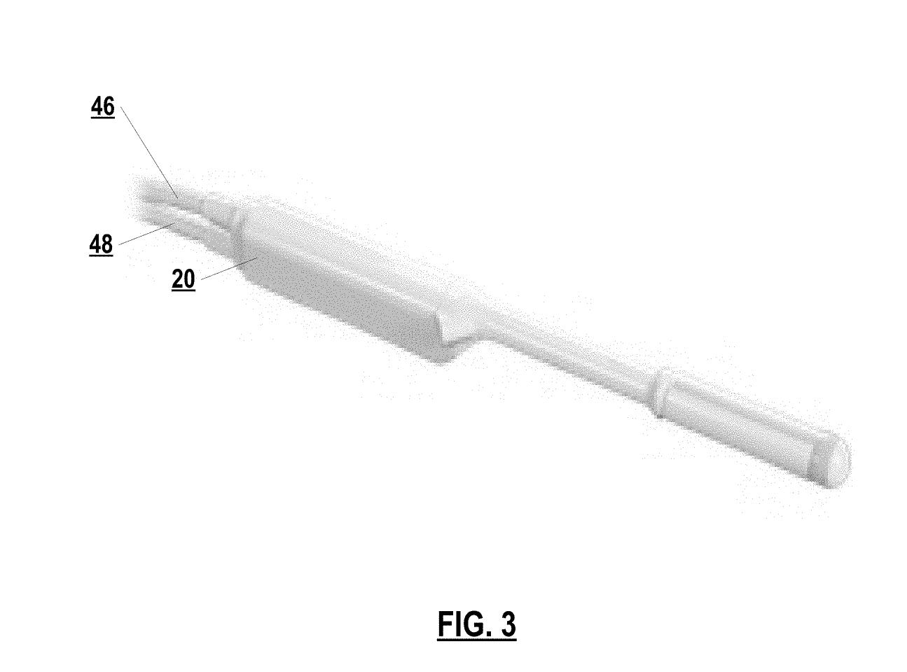

The probe 20 connects to a processor unit 52 (FIG. 3) via a cable 36. The probe includes a controller 34 to enable and control the electronically steerable emitter 30 and the electronically steerable receiver 32. The processor unit 52 instructs the electronically steerable emitter 30 via a first signal to emit a laser pulse to excite a prostate cancer infused with a fluorophore.

As the fluorophore changes state, the fluorophore emits a radio frequency energy due to a photoacoustic effect signal due to transient thermoelastic expansion. The radio frequency energy is received by the electronically steerable receiver 32 and returns a second signal to the processor unit 52.

The second signal is analyzed by the processor unit 52, for example, using digital signal processing and/or other algorithms to map the prostate and the prostate cancer. The processor can use at least one of a graphics processing unit (GPU) and one or more computer processors of a computing system to interpolate said transient thermoelastic expansion and produce a voxel representation of the prostate and an area proximate the prostate.

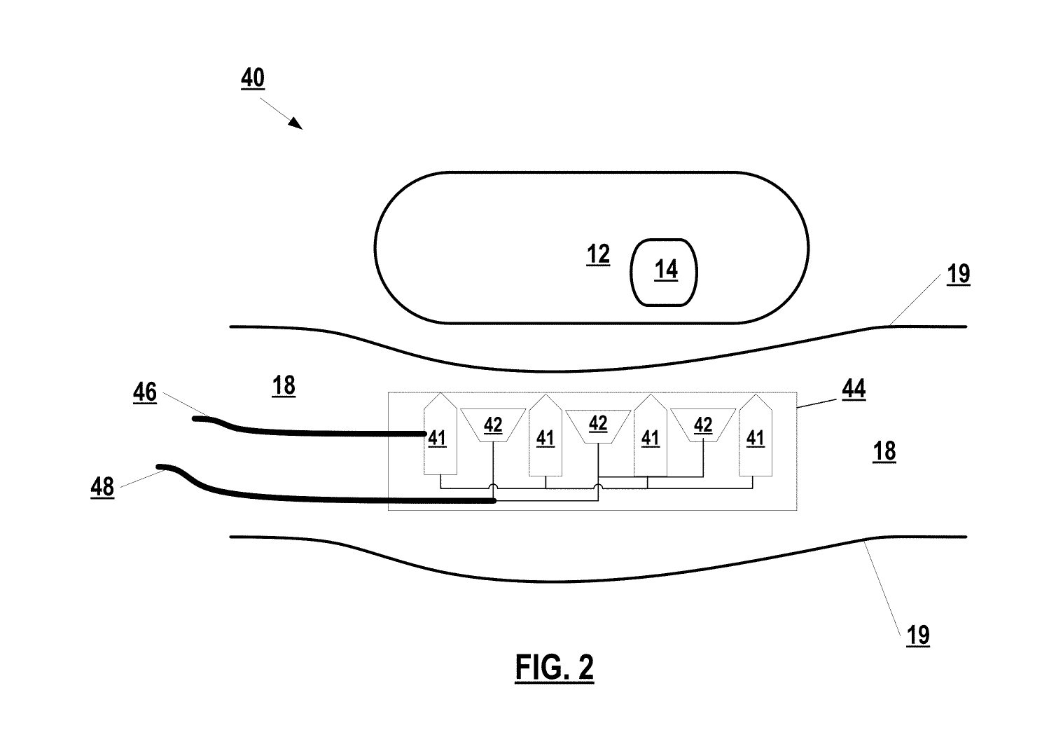

FIG. 2 is schematic diagram of a photoacoustic probe detection system 40. The prostate 12 is shown with a cancerous region 14. A second probe 44 is inserted into a colon 19 going through a rectum 18 and placed adjacent to the prostate 12. The second probe 44 has an arrangement of fiber optic emitters 41 and connects to a processor unit 52 (FIG. 3) via a fiber optic cable 46. The processor unit 52 generates a laser pulse via an external light module to be transmitted over the fiber optic cable 46 and emitted via the fiber optic emitters 41 to excite a prostate cancer infused with a fluorophore.

As the fluorophore changes state, the fluorophore emits a radio frequency energy due to transient thermoelastic expansion. The radio frequency energy is received by a photoacoustic sensor 42 and returns a second signal to the processor unit 52.

The second signal is analyzed by the processor unit 52, for example, using digital signal processing and/or other algorithms to map the prostate and the prostate cancer. The processor can use at least one of a graphics processing unit (GPU) and one or more computer processors of a computing system to interpolate said transient thermoelastic expansion and produce a voxel representation of the prostate and an area proximate the prostate.

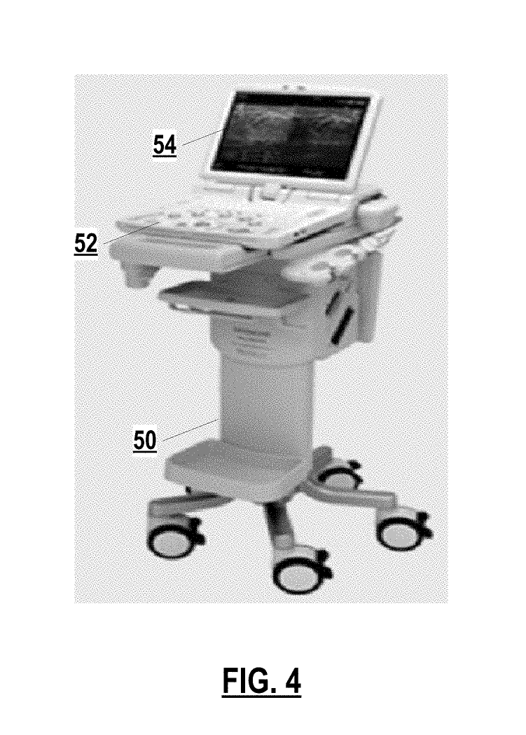

FIG. 3 illustrates a photoacoustic probe 44 showing the fiber optic cable 46 and a second signal cable 48 and FIG. 4 illustrates photoacoustic system implemented on a transportable cart 50. The transportable cart 50 holds a processor unit 52 and a display 54.

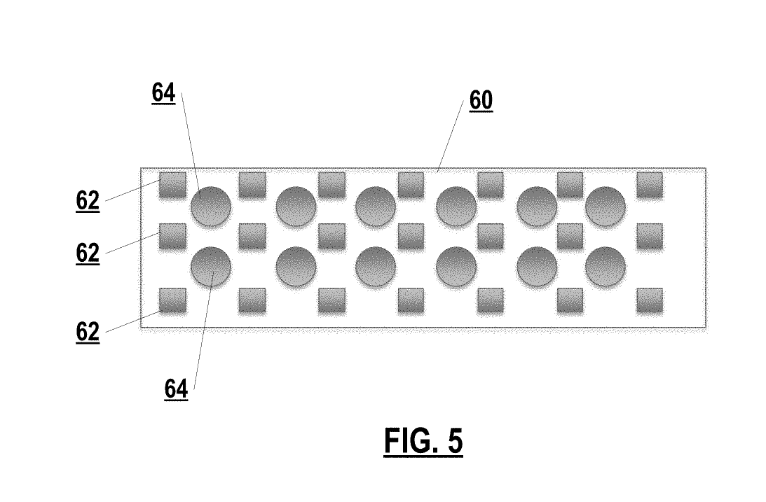

FIG. 5 illustrates a front side (that presses against prostate) showing detail of a photoacoustic probe head with an arrangement of integrated fiber optics transmitters 64 and an arrangement of ultrasound receivers 62.

The photoacoustic probe detection system 10 is deployed after a fluorophore peptide dye conjugate compound which has at least one absorption wavelength in a range of 380 to 1400 nm is injected into the patient, for example, 700 nanometer wherein said compound attaches to a prostate-specific membrane antigen (PSMA) expressed by a prostate cancer cell. A photo-acoustic imaging probe 20, having an operative end configured for scanning a prostrate, said photo-acoustic imaging probe to be inserted in at least one of a rectum, urethra, or placed proximal the prostate.

The photo-acoustic imaging probe 20 has an emitter 30 to emit a first signal towards the prostrate and a prostate cancer cell to excite said fluorophore peptide dye conjugate compound. The probe 20 has a receiver 32 to receive a second signal from said fluorophore peptide dye conjugate compound, thereby indicating a cancerous region of the prostrate. The processor unit 52 connected to said probe 20, wherein said processor unit 52 is configured and operable for receiving and processing said second signal to produce a tomographic representation of said prostrate. The processor unit 52 contains at least a processor, a read memory, a read-write memory, an instruction set and an interface.

In an embodiment, the first signal is produced in processor unit 52 and fiber optically coupled to the photo-acoustic imaging probe 44, wherein said first signal is generated by at least a laser, and a photo diode.

In another embodiment, the first probe 20 or the the photo-acoustic imaging probe 44 comprise at least one of a phased array of ultrasound sensors, a set of monolithic single channel ultrasound sensors, and an ensemble of phased array of ultrasound sensors.

The processor unit 52 can contain at least one of a graphics processing unit (GPU) and one or more computer processors of a computing system to interpolate said transient thermoelastic expansion and produce a voxel representation of the prostate and an area proximate the prostate. The processor unit 52 can also contain a memory unit to store said voxel representation of the prostate and the area proximate the prostate.

The processor unit 52 is communicatively coupled to a screen, a virtual reality display mechanism, and an augmented reality display mechanism to display a visual representation of the prostate and the cancer, for example a 3-D tomographic representation.

The fluorophore peptide dye conjugate can be compounded to attach to a cancerous region in at least one of a breast, a lung, a bronchus, a colorectal region, a uterine corpus, a bladder and a thyroid. Additionally, the fluorophore peptide dye conjugate can be compounded to attach to attach to a cavernous nerve adjacent the prostate, thereby the cavernous nerve to be differentiated from the prostrate and the cancerous region.

Furthermore, a fluorophore peptide dye conjugate can be compounded to attach to a medically important cell in a medically important region of interest in at least one of a heart region, a brain region, a chest region, a stomach region, a leg region, an arm region, and a head region, thereby a detection of nerves is feasible due to a long path length of light, and a low scattering of said second signal. Additionally, a nerve fluorophore peptide dye conjugate can be compounded to attach to attach to a nerve cell in at least one of a breast, a lung, a heart, a bronchus, a colorectal region, a uterine corpus, a bladder, a thyroid and any corpus part.

In another embodiment, the photoacoustic imaging system 10 is configured and operable with high frequency ultrasound (HIFU) cancer ablation system to ablate said cancer region via a feedback loop. For example, until said photoacoustic imaging system 10 determines that a voxel cancer value is less than less than 5 percent of an initial cancer voxel value associated.

In another embodiment, the photoacoustic imaging system 10 obtains measurements of the prostrate with the probe before injecting the fluorophore peptide dye conjugate compound to obtain a first set of baseline data of the prostrate. Then obtaining measurements of the prostrate with the probe after injecting the fluorophore peptide dye conjugate compound to obtain a second set of data of the prostrate. A prostate image is obtained by subtracting the first set of baseline data from the second set, thereby producing a differential image showing only the cancerous tissues.

In another embodiment, the photoacoustic imaging system 10 includes a 3-D coordinate orientation sensor for determining a location and orientation of the probe using.

Peptides for Both Visual and Photoacoustic Nerve Identification

Peptides can carry fluorophores to nerve cells. This means, for example, during a traditional prostatectomy, nerves could be visualized and over laid on the surgical field of view. In an embodiment of the present invention, a different fluorophore be used to give a different spectral output, so nerves and prostate cancer can be differentiated. That is, in use, one could pulse with one color laser to illuminate on nerve fluorophore, record the photoacoustic or visual image, then pulse with a different laser to excite the fluorophore on the PSMA.

Thus an embodiment of the present invention is to have photo-acoustic identification of the nerves as well as the cancer. For example, a fluorophore is excited with near infrared light. An ultrasound detector (not shown) is deployed to receive the signals. An advantage to this embodiment is this allows much greater depth penetration, and allows cancers to be seen and identified that are out of visual field.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

What is claimed is:

1. An apparatus for use in a minimally invasive prostate cancer detection system, the apparatus comprising:
a fluorophore peptide dye conjugate compound which has at least one absorption wavelength in a range of 380 to 1400 nm, wherein said compound attaches to a prostate-specific membrane antigen (PSMA) expressed by a prostate cancer cell;
a photo-acoustic imaging probe having an operative end configured for scanning a prostrate, said photo-acoustic imaging probe to be inserted in at least one of a rectum, urethra, or placed proximal the prostate, said photo-acoustic imaging probe comprising:
an emitter to emit a first signal at the prostrate and a prostate cancer cell and excite said fluorophore peptide dye conjugate compound;
a receiver to receive a second signal from said fluorophore peptide dye conjugate compound, thereby indicating a cancerous region of the prostrate; and
a processor unit connected to said probe, wherein said processor unit is configured and operable for receiving and processing said second signal to produce a tomographic representation of said prostrate, wherein the processor unit contains at least a processor, a read memory, a read-write memory, an instruction set and an interface.

2. The apparatus according to claim 1, wherein the first signal is produced in at least the photo-acoustic imaging probe, and an external light module and fiber optically coupled to the photo-acoustic imaging probe, wherein said first signal is generated by at least a laser, and a photo diode.

3. The apparatus according to claim 1, wherein said second signal is a photoacoustic effect signal created by a transient thermoelastic expansion by the absorption of said first signal of the fluorophore peptide dye conjugate compound.

4. The apparatus from claim 3 comprises at least one of a phased array of ultrasound sensors, a set of monolithic single channel ultrasound sensors, and an ensemble of phased array of ultrasound sensors.

5. The apparatus of claim 3, wherein the processor unit comprises:
at least one of a graphics processing unit (GPU) and one or more computer processors of a computing system to interpolate said transient thermoelastic expansion and produce a voxel representation of the prostate and an area proximate the prostate;
a memory unit to store said voxel representation of the prostate and the area proximate the prostate; and
at least one of a screen, a virtual reality display mechanism, and an augmented reality display mechanism to display a visual representation of the prostate and the cancer.

6. The apparatus of claim 1, wherein said fluorophore peptide dye conjugate is compounded attaches to a cancerous region in at least one of a breast, a lung, a bronchus, a colorectal region, a uterine corpus, a bladder and a thyroid.

7. The apparatus of claim 1, wherein a nerve fluorophore peptide dye conjugate is compounded to attach to a cavernous nerve adjacent the prostate, thereby the cavernous nerve to be differentiated from the prostrate and the cancerous region.

8. The apparatus of claim 1, wherein said fluorophore peptide dye conjugate is compounded to attach to attach to a medically important cell in a medically important region of interest in at least one of a heart region, a brain region, a chest region, a stomach region, a leg region, an arm region, and a head region, thereby a detection of nerves is feasible due to a long path length of light, and a low scattering of said second signal.

9. The apparatus of claim 8, wherein a nerve fluorophore peptide dye conjugate is compounded to attach to attach to a nerve cell in at least one of a breast, a lung, a heart, a bronchus, a colorectal region, a uterine corpus, a bladder, a thyroid and any corpus part.

10. The apparatus of claim 8, wherein the photo-acoustic imaging probe is configured and operable with high frequency ultrasound (HIFU) cancer ablation system to ablate said cancer region via a feedback loop until said apparatus determines that a voxel cancer value is less than less than 5 percent of an initial cancer voxel value associated.

11. A method to minimally invade and detect prostate cancer, the method comprising:
injecting a fluorophore peptide dye conjugate compound into a subject, wherein said compound attaches to a cancerous region in a prostrate;
inserting a probe inside a colon, said probe having with an operative end configured for scanning the prostrate;
emitting a first signal towards the prostrate and exciting said fluorophore peptide dye conjugate compound;
receiving a second signal from a photoacoustic effect signal created by a transient thermoelastic expansion by an absorption of said first signal of the fluorophore peptide dye conjugate compound; and
processing said second signal in a processor unit, thereby producing a 3-D tomographic representation of said prostrate.

12. The method of claim 11 in which the conjugate compound has at least one absorption wavelength in a range of 380 to 1400 nm.

13. The method of claim 11, further comprising storing at least a set of the second signal from the probe, and the tomographic representation in a memory unit of the processor unit and displayed visually.

14. The method of claim 13, further comprising:
obtaining measurements of the prostrate with the probe before injecting the fluorophore peptide dye conjugate compound to obtain a first set of baseline data of the prostrate; and
obtaining measurements of the prostrate with the probe after injecting the fluorophore peptide dye conjugate compound to obtain a set of differential data of the prostrate; and
creating a prostate image by subtracting the first set of baseline data from the set of differential data, thereby producing a difference image showing the prostate cancer.

15. The method of claim 13, further comprising determining a location and orientation of the probe using a 3-D coordinate orientation sensor.

16. The method of claim 13, further comprising, compounding said fluorophore peptide dye conjugate compound to attach to attach to said cancerous region in at least one of a breast, a lung, a heart, a bronchus, a colorectal region, a uterine corpus, a bladder and a thyroid.

17. The method of claim 13, further comprising, compounding a nerve fluorophore peptide dye conjugate is compounded to attach to attach to a cavernous nerve adjacent the prostate, thereby the cavernous nerve to be differentiated from the prostrate and the cancerous region.

18. The method of claim 13, wherein a nerve fluorophore peptide dye conjugate is compounded to attach to attach to a nerve cell in at least one of a breast, a lung, a heart, a bronchus, a colorectal region, a uterine corpus, a bladder and a thyroid.

19. The method of claim 13, further comprising, attaching a high frequency ultrasound (HIFU) cancer ablation system and operating in a feedback loop until said HIFU cancer system ablates a cancerous region until that a voxel cancer value is less than less than 5 percent of an initial cancer voxel value.

20. A minimally invasive prostate cancer detection system, comprising:
means for injecting a fluorophore peptide dye conjugate compound into a subject, wherein said compound attaches to a cancerous region in a prostrate;
means for inserting a probe inside a colon, said probe having with an operative end configured for scanning the prostrate;
means for emitting a first signal towards the prostrate and exciting said fluorophore peptide dye conjugate compound;
means for receiving a second signal from a photoacoustic effect signal created by a transient thermoelastic expansion by an absorption of said first signal of the fluorophore peptide dye conjugate compound;
means for processing said second signal in a processor unit; and
means for producing a 3-D tomographic representation of said prostrate and the cancer in at least one of a screen, a virtual reality display mechanism, and an augmented reality display mechanism.
